(19) United States
(12) Patent Application Publication
(10) Pub. No.: US 2011/0256184 A1
(43) Pub. Date: Oct. 20, 2011

(54) Non-ordered Mesoporous Silica Structure for Biomolecule Loading and Release

(75) Inventors: Chenghong Lei, Richland, WA (US); Jun Liu, Richland, WA (US); Xiaolin Li, Richland, WA (US)

(73) Assignee: BATTELLE MEMORIAL INSTITUTE, Richland, WA (US)

(21) Appl. No.: 13/085,674

(22) Filed: Apr. 13, 2011

Related U.S. Application Data
(60) Provisional application No. 61/323,966, filed on Apr. 14, 2010.

(51) Int. Cl. A61K 39/395; C07F 7/02; C01B 33/00; A61K 9/00; C01B 33/12

(52) U.S. Cl. 424/400; 424/130.1; 424/158.1; 424/173.1; 423/335; 423/325; 556/437

(57) ABSTRACT

A non-ordered geometric mesoporous structure that provides for enhanced loading of antibodies such as IgG as compared to ordered mesoporous structures. This structure is formed by treating silica precursors at a hydrothermal aging temperature between 100 and 200 degrees C. This creates the non-ordered mesoporous structure. Biomolecules such as IgG can then be spontaneously loaded via non-covalent bonding within the as-made or functionalized mesoporous structure.

### CLAIM TO PRIORITY

This application claims priority from Application Ser. No. 61/323,966 filed Apr. 14, 2010 entitled Functionalized Nano/micromaterials for medical therapies. The contents of which are hereby incorporated by reference.

### STATEMENT AS TO RIGHTS TO INVENTIONS MADE UNDER FEDERALLY-SPONSORED RESEARCH AND DEVELOPMENT

This invention was made with Government support under Contract DE-AC0576RLO1830 awarded by the U.S. Department of Energy as well as Grant R01GM080987 from the National Institutes of Health. The Government has certain rights in the invention.

### BACKGROUND OF THE INVENTION

Mesoporous silica possess various unique features such as large surface areas, controllable porous structures, versatile functionalization accessibilities, and are suitable for scalable productions. As such, they have been contemplated as carriers for delivery of a variety of materials including proteins, nucleic acids, and various drug molecules. Such materials can be entrapped within the mesoporous silicas via non-covalent interaction, and then released from the mesopores. The intramesoporous structures, i.e. pore size (diameter), surface area and pore volume influence the capacity of the material for drug loading and release. Traditionally it has been believed that higher levels of loading and of functional efficacy are obtained as the ordered nature of the pores increases. While this is true in some respects, additional ways of forming structures that provide increased ability to perform the drug holding and release functions. What is needed therefore is a structure and a method for making a structure that provides these advantages. The present invention meets these needs.

### SUMMARY

The present invention is a non-ordered/disordered open mesoporous structure, that is, a mesocellular structure, that provides for enhanced loading of antibodies such as IgG as compared to ordered mesoporous structures. This structure is formed by treating silica precursors at a hydrothermal aging temperature (HAT) between 80 and 200 degrees C. to create a non-ordered mesoporous structure with a large pore opening as determined by the desorption pore size using the Joyner-Halenda (BJH) method. This is contrary to prior art configurations which teach methods to increase the order and uniformity of the pores in a mesoporous material. The present invention utilizes an optimum temperature to create structures with disorder/non-ordered pores with widest pore openings. At a lower temperature, regular, ordered mesoporous silica is formed. At a much higher temperature, the pore structure is more disordered, forming the mesocellular structure:

As described in the detailed description these structures enable biomolecules such as Immunoglobulin G (IgG)-type antibodies to be spontaneously (without the use of a covalent linker) loaded within the structure via non-covalent interactions. In some instances the surfaces of the non-ordered mesoporous silica can also be functionalized, through a process such as carboxylethyl-functionalization or aminopropyl functionalization. In some preferred embodiments the biomolecule is a IgG-type antibody targeted to an antigen selected from the group consisting of CD137; CTLA-4; CD3; CD83; CD25; CD28; CD40; 4-1BB; GITR; Her-2; TGFβ; OX-40; TGFα or any other antigen. The purpose of the foregoing abstract is to enable the United States Patent and Trademark Office and the public generally, especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Various advantages and novel features of the present invention are described herein and will become further readily apparent to those skilled in this art from the following detailed description. In the preceding and following descriptions we have shown and described only the preferred embodiment of the invention, by way of illustration of the best mode contemplated for carrying out the invention. As will be realized, the invention is capable of modification in various respects without departing from the invention. Accordingly, the drawings and description of the preferred embodiment set forth hereafter are to be regarded as illustrative in nature, and not as restrictive.

### BRIEF DESCRIPTION OF THE DRAWINGS

FIGS. 1a and 1b show transmission electron microscopy (TEM) images of AMS-100 and AMS-120.

FIG. 1c is a graph showing the relationship of pore sizes and temperature

FIG. 1d shows the trends of Brunauer-Emmett-Teller (BET) surface areas and pore volumes of AMSs with varied temperatures.

FIG. 2a shows the protein loading density (P_{LD}) of Rat IgG in pH 7.4, 10 mM phosphate/0.14 M NaCl (PBS).

FIG. 2b shows the protein structures of IgG with surface charge distribution.

FIG. 2c shows the fluorescence emission (excitation=278 nm) of IgG in 20% HOOC-FMS-120 at different P_{LD} in pH 7.4, PBS. [protein]=10 μg/mL.

FIG. 2d shows the protein loading density of GOX in pH 7.4, 10 mM sodium phosphate.

FIG. 2e shows the protein structures of GOX with surface charge distribution.

FIG. 2f shows the fluorescence emission of GOX in 20% NH₂-FMS-120 at different P_{LD} in pH 7.4, 1.0 mM sodium phosphate. [protein]=10 μg/mL.

FIG. 2g shows the protein loading density of GI in pH 7.4, PBS.

FIG. 2h shows the protein structure of GI with surface charge distribution.

FIG. 2i shows the fluorescence emission of GI in 20% NH₂-FMS-120 at different P_{LD} pH 7.4, PBS. [protein]=10 μg/mL.

### DETAILED DESCRIPTION OF THE INVENTION

The following description includes the preferred best mode of one embodiment of the present invention. It will be clear from this description of the invention that the invention is not limited to these illustrated embodiments but that the invention also includes a variety of modifications and embodiments thereto. Therefore the present description should be seen as illustrative and not limiting. While the invention is susceptible of various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

In one example of the invention, as-made mesoporous silica (AMS) was created by controlling the hydrothermal aging temperature (HAT) for the silica gel prior to its calcination. As described hereafter, we found that HAT had a critical effect on the intramesoporous structure and thus on the protein loading and release. The larger desorption pore size allowed the larger protein loading for immunoglobulin G (IgG) and glucose isomerase (GI), while the larger surface area resulted in the larger protein loading for glucose oxidase (GOX).

As-made mesoporous silicas, were prepared by dissolving 4.0 g of Pluronic P-123 (MW=5,800) in 2 M HCl solution (1.20 mL) at 35-40° C. Then 6.0 g of mesitylene and 8.5 g of tetraethylorthosilicate (TEOS) was added to the milky solution and stirred for 6 h at the same temperature. The mixture was then transferred into a Teflon-lined autoclave container and heated up to the desired temperature (80-200° C.) for 24 h. The white precipitate was then collected by filtration, dried in air, and finally calcined at 550° C. for 6 hours. This AMS material was then functionalized in several ways.

In a typical synthesis for 20% NH₂-FMS or 20% HOOC-FMS, 0.5 g of the AMS described above was first suspended in toluene (25 mL) and pretreated with water (0.16 mL) in a three-necked 125 mL round-bottom flask, which was fitted with a stopper and reflux condenser. This suspension was stirred vigorously for 2 h to distribute the water throughout the mesoporous matrix, during which time it became thick and homogeneous slurry. At this point, a corresponding amount of 3-aminopropyltriethoxysilane (APTES, MW=221.37) or 2-cyanoethyl trimethoxysilane (CTS, MW=175.26) to silanize 20% of the total available silanol groups (5×10¹⁸ silanol groups per square meter) of AMS was added and the mixture was refluxed at 120° C. for 6 h.

The mixture was allowed to cool to room temperature and the product was collected by vacuum filtration. The resulting NH₂-FMSs or NC-FMSs were washed with ethyl alcohol repeatedly and dried under vacuum. To hydrolyze cyano groups, 10 mL of 50% of H₂SO₄ solution was added to the mixture and stirred in an ice-bath for 3 h. The resulted HOOC-FMSs were filtered, washed with water extensively, and dried in vacuum. In this work, AMS prepared at HATs 80° C., 100° C., 110° C., 120° C. and 130° C. are termed as AMS-80, AMS-100, AMS-110, AMS-120 and AMS-130 and correspondingly FMSs as FMS-80, FMS-100, FMS-110, FMS-120 and FMS-130, respectively.

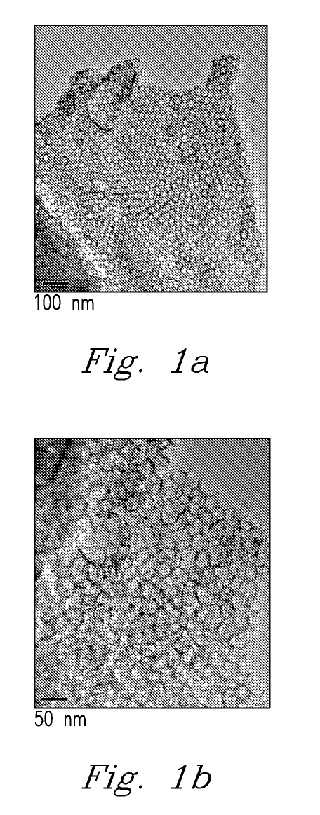

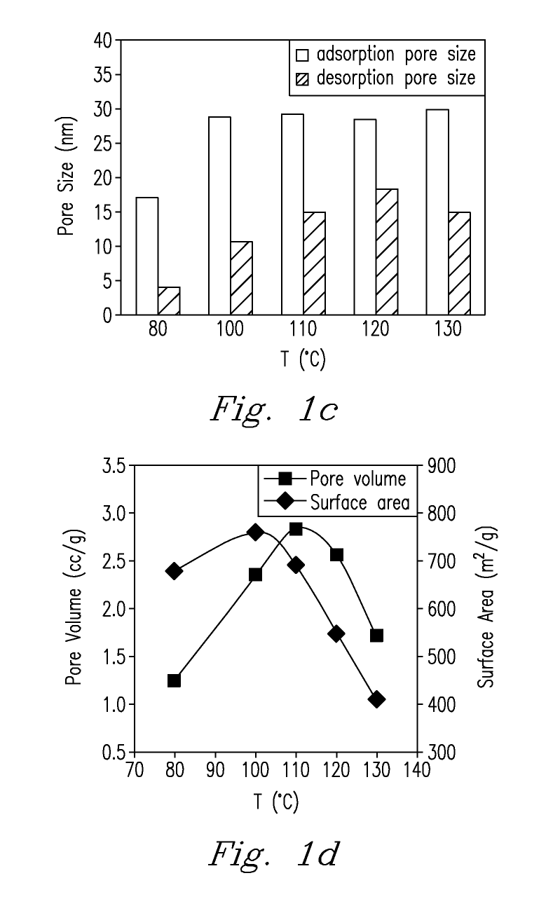

FIGS. 1a and 1b show transmission electron microscopy (TEM) images of AMS-100 and AMS-120 respectively. Although both AMS-100 and AMS-120 display the similar adsorption pore sizes of ˜30 nm, AMS-100 has an ordered mesopore structure while AMS-120 has a large degree of disordered pores. Nevertheless, AMS-120 still reveals a more or less uniform cage-like porous structure. AMSs prepared in this manner have a mesocellular foam-like intramesoporous structure with relatively large cages connected by narrow pore entrances. The adsorption pore size corresponds to the wide cage diameter while the desorption pore size is related to the narrow pore entrance size. FIG. 1c shows the effect of the temperature on the pore size and FIG. 1d shows the results from N₂ sorption measurements that AMS-80 has a Barrett-Joyner-Halenda (BJH) adsorption pore size of ˜17 nm and a BJH desorption size of ˜3.9 nm, while all other AMSs have very close adsorption pore sizes of ˜30 nm but the desorption size increased from ˜1.0 to 1.8 nm with the increased HATs from 100° C. to 120° C. and then decreased at 130° C. However, the Brunauer-Emmett-Teller (BET) surface areas and pore volumes of AMSs show the different trends with varied HATs (FIG. 1d). The surface areas were increased from ˜300 to 800 m²/g with the increased HATs from 80° C. to 100° C. and then decreased from 100° C. to 130° C. (FIG. 1d), while the pore volumes were increased from ˜1.25 to 2.84 cm³/g with the increased HATs from 80° C. to 110° C. and then decreased from 1.1.0° C. to 130° C. (FIG. 1d).

To study the intramesoporous structure effects on protein loading and release, AMSs and FMSs were tested with the neutral protein rat IgG (M. W. 150 kDa) with a Y-like molecular shape, one charged protein cox (M. W. 160 kDa) with an elliptical molecular shape, and another charged protein GI (M.W. 1.73 kDa) with a spindle-like molecular shape. In a standard procedure, ˜1 mg of AMS or FMS was incubated with ˜0.4 mg of the protein in pH 7.4, PBS or 10 mM sodium phosphate, where the protein would be spontaneously entrapped in the mesopores. We defined the protein loading density (P_{LD}) as the protein amount (μg or mg) entrapped with 1 mg of FMS.

An aliquot of 1.0-2.0 mg of the prepared AMS or FMS was then placed in a 1.5-mL tube for incubation with 200-400 μL of a protein stock solution in pH7.4, PBS. 0.4 mg protein was used for incubation per mg of mesoporous silica. The incubation was carried out at 21° C. shaking at 1400 min⁻¹ on an Eppendorf Thermomixer 5436 for 24 h. The protein stock in the absence of mesoporous silica was also shaken under the same conditions for comparison. The mesoporous silica-protein composite was separated by centrifugation and the first supernatant (the elution number: 0) was removed. The amounts of proteins were measured by Bradford method using bovine gamma globulin as standards. To test the in vitro gradual release of the proteins from mesoporous silica, 200 μl of a simulated body fluid that has ion concentrations nearly equal to those of human blood plasma (buffered at pH 7.4 with 50 mM Tris-HCl) as the elution buffer (the elution number: 1) was added, and shaken with the mesoporous silica-protein composite for 5 minutes and then the supernatant was separated by centrifugation. For each subsequent elution with the same elution buffer, the mesoporous silica-protein composite was repeatedly separated by centrifugation and the amount of the released protein in the supernatant was measured by UV at 280 nm using the diluted protein stock solutions as standards.

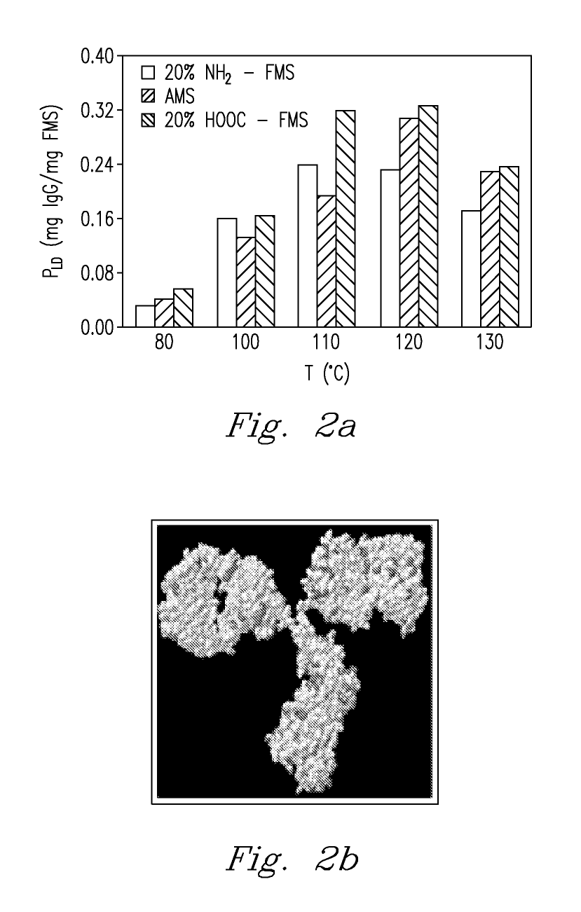

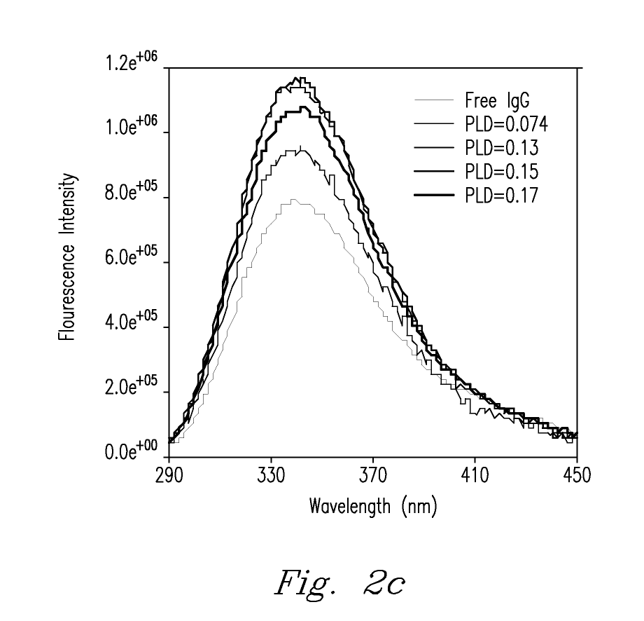

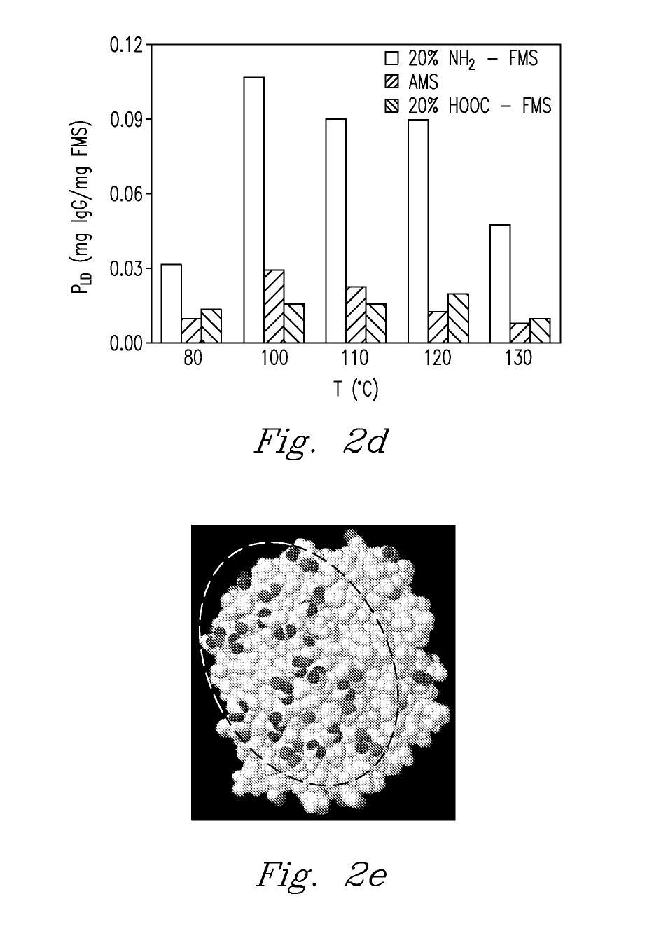

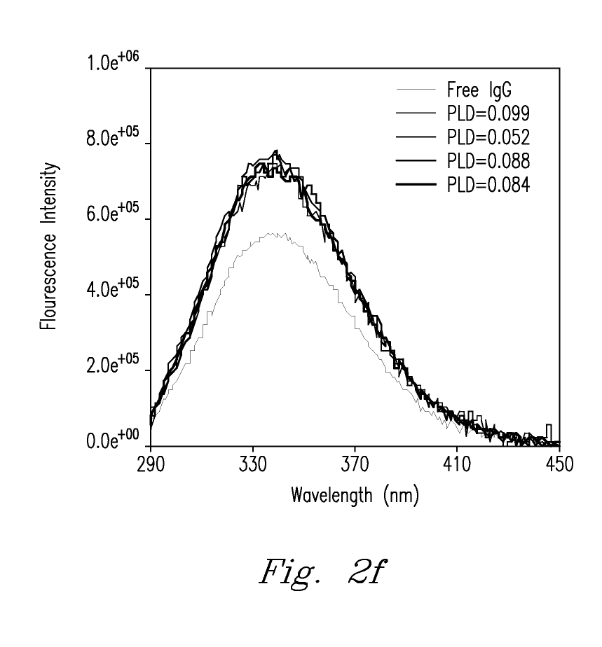

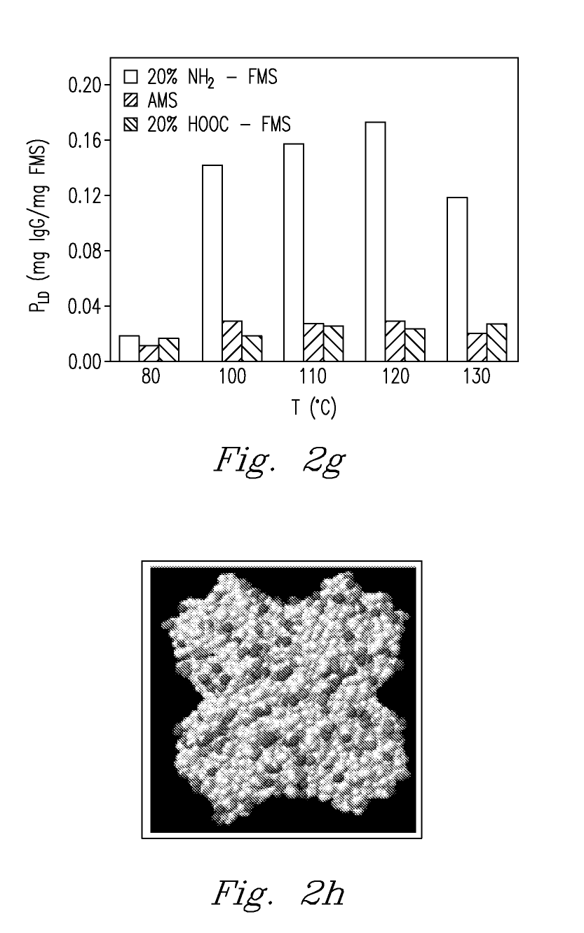

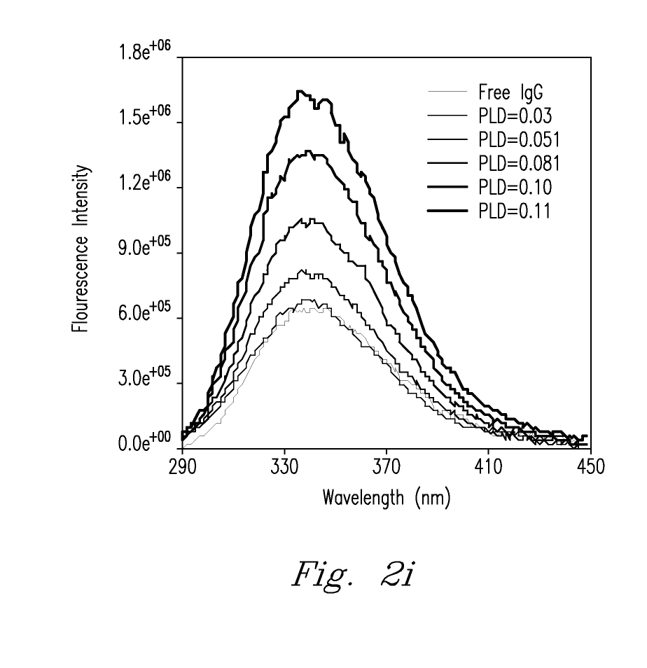

FIG. 2a shows the protein loading density (P_{LD}) of Rat IgG, FIG. 2(d) shows the protein loading density of GOX, and FIG. 2(g) shows the protein loading density of GI in AMS, 20% NTH-FMS, and 20% HOOC-FMS at different HATs. FIG. 2b shows the protein structures of IgG, FIG. 2e shows the protein structures of GOX, and FIG. 2h shows the protein structure of GI with surface charge distribution. FIG. 2c shows the fluorescence emission (excitation=278 nm) of IgG in 20% HOOC-FMS-120, FIG. 2f shows the fluorescence emission of GOX and FIG. 2i shows the fluorescence emission of GI in 20% NH₂-FMS-120 at different P_{LD}. pH 7.4, PBS was used as the working buffer for IgG and GI, pH 7.4, 10 mM sodium phosphate for GOX. [protein]=10 μg/mL.

FIG. 2a displays IgG loading density in AMS, 20% NH₂-FMS and 20% HOOC-FMS. AMS-120 and FMS-120 samples had the highest protein loading density while AMS-80 and FMS-80 ones had the lowest loading density. The IgG loading density in AMS and FMS changed with the varied desorption pore size corresponding to HAT at which AMS was prepared (FIGS. 1c and 2a). Since AMS and FMS samples prepared at HAT 100-130° C. have the similar adsorption size (˜30 nm), it was the desorption pore size (the narrow pore entrance size) that played a dominant role governing the protein loading density of IgG in AMS and FMS (FIG. 2a), that is, the smaller desorption pore size limited the protein to be entrapped inside the pores presumably due to the steric hindrance of IgG's Y-like shape (FIG. 2b) formed from 4 peptide chains. The surface area of AMS and FMS was not a prominent role affecting the IgG loading density because AMS-100 and FMS-100 had the largest surface area (FIG. 1d) but the AMS-120 and FMS-120 displayed the largest loading density (FIG. 2a).

The fluorescence emission of IgG in FMS at different Pin was compared to the free IgG at the excitation wavelength of 278 nm, allowing excitation of both tyrosinyl and tryptophanyl residues. Comparing the free IgG to FMS-IgG (FIG. 2c), there was no dramatic emission peak shift but increased emission intensity at ˜340 nm because of the interaction of IgG with FMS along the occupancy of the mesopore surface. When P_{LD} of IgG in FMS was increased to 0.17 mg/mg of FMS (FIG. 1.c), the fluorescence intensity of IgG in FMS was decreased and closer to the emission spectra of the free IgG comparing to that of the lower P_{LD} of IgG in FMS (FIG. 2c). After all the available silica surface was fully occupied with the monolayer IgG molecules, the neutral IgG molecules kept aggregating in FMS with no more direct attachment as long as the pore volume allows, and thus resulted in the fluorescence intensity closer to the free IgG in the aqueous environment.

FIG. 2d shows GOX loading density in AMS and FMS. At pH 7.4, GOX is a negatively charged protein. The negatively charged protein would be largely loaded in the positively-charged mesoporous silica but be expelled from loading inside the negatively-charged mesoporous silica. As expected, the GOX loading density was very low in AMS and 20% HOOC-FMS but much higher in 20% NH₂-FMS (FIG. 2d). Comparing NH₂-FMSs, NH₂-FMS-80 had the relatively lowest GOX loading density and NH₂-FMS-100 has the highest loading density. The GOX loading density decreased from NH₂-FMS-100 to NH₂-FMS-130, indicating that the GOX loading density in NH₂-FMS changed with the varied surface area corresponding to HAT at which AMS was prepared (FIGS. 1d and 2d). It was the surface area that played the prominent role governing the protein loading density of GOX in AMS and FMS, that is, the larger positively-charged surface area allowed the larger amount of the negatively-charged protein to be accommodated. For the elliptical GOX (FIG. 2e), which has no steric hindrance when loading in NH₂-FMS, the larger surface area is needed to ensure higher protein loading density. To study the possible entrapping states of GOX in FMS, we also studied the fluorescence emission of GOX in FMS at different P_{LD} comparing to the free GOX at the excitation wavelength of 278 nm. Similarly to FMS-IgG (FIG. 2c), we also found that there was no dramatic emission peak shift but increased emission intensity at ˜340 nm because of the interaction of GOX with FMS along the electrostatic monolayer occupancy of the mesopore surface (FIG. 2f). However, even when P_{LD} of GOX in FMS was increased to the largest P_{LD} (FIG. 2d), its fluorescence intensity was still similar to that of GOX in FMS with the lower P_{LD}, which was still far from the emission spectra of the free IgG (FIG. 2f). Therefore, after all available silica surface was fully occupied with the monolayer GOX molecules, unlike IgG, the charged GOX molecules would not be able to accumulate in FMS due to the electrostatic expulsion and the limit of pore volumes. GOX consists of two identical monomers. Each monomer of the dimeric GOX molecule are asymmetrically interfaced with one same region with much more concentrated negative charges than other regions. For the whole GOX molecule, we believe that this negatively region (circled area in FIG. 2e) of one monomer would be attached to the mesoporous wall while the same region of the other monomer was pointed toward the inside axis of the mesopores. This way would provide the electrostatically expulsive microenvironment that can prevent GOX from further accumulation inside the mesopores after the monolayer occupancy.

FIG. 2g shows GI loading density in AMS and FMS. At pH 7.4, GI is a negatively charged protein. As expected, GI loading density was very low in AMS and 20% HOOC-FMS but much higher in 20% NH₂-FMS (FIG. 2g), that is, a similar electrostatic selectivity to GOX. Comparing NH₂-FMSs, however, NH₂-FMS-120 had the highest loading density for GI. The GI loading density decreased from NH₂-FMS-120 to NH₂-FMS-130, indicating that the GI loading density in NH₂-FMS changed with the varied desorption pore size corresponding to HAT at which AMS was prepared (FIGS. 1c and 2g). It was the desorption pore size that played the prominent role governing the protein loading density of GI in NH₂-FMS, that is, the smaller desorption pore size limited the protein to be entrapped inside the pores presumably due to the steric hindrance of GI's spindle-like shape (FIG. 2h). Fluorescence emission of GI in NH₂-FMS at different loading densities were also compared to the free GI at the excitation wavelength of 278 nm. Similarly to FMS-GOX (FIG. 2f), we also found that there was no dramatic emission peak shift but increased emission intensity at ˜340 nm because of the interaction of GI with FMS. We noticed that the fluorescence intensity was increased along the electrostatic monolayer occupancy of the mesopore surface (FIG. 2h) with the increasing P_{LD} of GI in FMS, similar to that of IgG in HOOC-FMS. Although both GOX and GI are the negatively charged proteins which have specific electrostatic selectivity for NH₂-FMS, but GI has a stronger hydrophobic interaction with NH₂-FMS since it had its relatively higher P_{LD} in pH 7.4, PBS containing 0.14 M NaCl. We believe that the stronger hydrophobic interaction of GI with NH₂-FMS resulted in the increased fluorescence intensity with the increasing P_{LD}. However, even when P_{LD} of GI was increased to the largest P_{LD} (FIG. 2g), its fluorescence intensity was still far from the emission spectra of the free IgG (FIG. 2i), while the fluorescence intensity of IgG had been decreased when the IgG loading density in FMS was still much less than its largest P_{LD} (FIGS. 2a and 2c). This differentiates the charged protein GI from the neutral protein IgG. We believe that, after all available silica surface was fully occupied with the monolayer GI molecules, similar to the charged GOX, the charged GI molecules would not be able to accumulate in FMS due to the electrostatic expulsion and the limit of pore volumes. This also explains why much higher loading densities of IgG were obtained than GOX and GI in FMS (FIGS. 2a, 2d and 2g) when the similar amount of the protein molecules were used for incubation with 1 mg of FMS. GI consists four identical monomers with nearly uniformly distributed net negative charges around the molecules (FIG. 2h), it is still not clear which region of the GI molecule was attached to the mesoporous wall.

As expected, all three proteins, IgG, GOX and GI were gradually released from AMS and FMS along the series of elutions using simulated body fluid because of their non-covalent interactions. In summary, for the Y-like IgG and the spindle-like GI, the larger desorption pore size allowed the larger protein loading, while for the elliptical GOX, the larger surface area resulted in the larger protein loading. Our results also showed that the neutral protein IgG can continue to aggregate in the mesopores after the monolayer occupancy, while the charged proteins GI and GOX were only attached inside the mesopores in a way of monolayers due to the electrostatic expulsion and the limit of pore volumes. The protein loading in AMS and FMS matters with the varied intramesoporous structure of mesoporous silicas with critical HATs as well as the biochemical structural characteristics of the protein (charge and shape). A clear understanding how intramesoporous structure effect the loading and release of proteins and other molecules would help the development of new mesostructured materials.

The present invention provides materials that can be utilized in a variety of applications including but not limited to drug and biomolecular delivery mechanisms. The biomolecules can be, for example, nucleic acids (e.g., single- or double-stranded DNA, cDNA, RNA, and PNA), antibodies (including antibody fragments, antibody conjugates), proteins (e.g., cytokines, enzymes, polypeptides, peptides), pharmaceuticals (such as vitamins, antibiotics, hormones, amino acids, metabolites and drugs), antigens, vaccines, and other biomolecules (such as ligands, receptors, viral vectors, viruses, phage or even entire cells) or fragments of these compounds, and the like, and any combinations thereof. In particular, biomolecules of particular interest are IgG antibodies directed toward antigens such as CD137; CTLA-4; CD3; CD83; CD25; CD28; CD40; 4-1BB; GITR; Her-2; TGFβ; OX-40; TGFα or any other antigen.

In some circumstances appropriate adaptation of the present invention may be necessary so as to address the particulars of the desired drug molecules. Such an embodiment may provide a controlled long-lasting release of a therapeutic drug at, for example an implant site that will allow much less dose and much longer dose intervals and thereby provide higher efficacy and fewer side effects and low costs as well.

As used herein, the term “antibody” includes, but is not limited to, polyclonal antibodies, monoclonal antibodies (mAb), human, humanized, chimeric antibodies (e.g., comprising an immunoglobulin binding domain, or equivalent, fused to another polypeptide) and any other subclasses or derivatives, and biologically functional antibody fragments sufficient for binding of the antibody fragment to the antigen of interest, such as single-chain variable fragment (scFv) fusion proteins, whether natural or partly or wholly synthetically produced, and derivatives thereof.

While various preferred embodiments of the invention are shown and described, it is to be distinctly understood that this invention is not limited thereto but may be variously embodied to practice within the scope of the following claims. From the foregoing description, it will be apparent that various changes may be made without departing from the spirit and scope of the invention as defined by the following claims.

What is claimed is:

1. A method for making a delivery device having at least one biomolecule entrapped in a non-ordered mesoporous substrate; said method characterized by the step of: treating silica precursors at a hydrothermal aging temperature between 100 and 200 degrees C. to create a non-ordered mesoporous geometric structure.

2. The method of claim 1 further comprising the step of functionalizing the surface of said mesoporous geometric structure.

3. The method of claim 1 wherein said biomolecule is an IgG antibody.

4. The method of claim 1 wherein said mesoporous silica precursor is selected from the group consisting of: silicas, clays, metal oxides, metal hydroxides, polymers, biopolymers, and polyelectrolytes.

5. The method of claim 1 wherein the mesoporous silica precursors are fused at a temperature between 115 and 130 degrees C.

6. The method of claim 2 wherein the functionalization step includes performing a carboxylic group-functionalization.

7. The method of claim 2 wherein the functionalization step includes performing an amino group-functionalization.

8. The method of claim 2 wherein the functionalization step includes performing a functionalization of using a group selected from the group consisting of HS—, HO₃S—, NC—, HO— and combinations thereof.

9. The method of claim 2 wherein said biomolecule is an antibody targeted to an antigen selected from the group consisting of CD137; CTLA-4; CD3; CD83; CD25; CD28; CD40; 4-1BB; GITR; Her-2; TGFβ; OX-40; TGFα or any other antigen.

10. A delivery device for IgG type antibodies comprising a mesoporous silica substrate defining a non-ordered pattern of pores.

11. The delivery device of claim 8 wherein said mesoporous silica substrate is as-made or non-functionalized.

12. The delivery device of claim 10 wherein said mesoporous silica substrate is functionalized.

13. The delivery device of claim 10 wherein said mesoporous silica substrate is as-made or non-functionalized.

14. A therapeutic delivery device characterized by a mesoporous silica substrate defining a non-ordered pattern of pores.
